(19) United States
(12) Patent Application Publication
(10) Pub. No.: US 2007/0175487 A1
(43) Pub. Date: Aug. 2, 2007

(54) Disposable Surgical Drape

(76) Inventor: J. Francois Eid, Larchmont, NY (US)

Correspondence Address: AMS RESEARCH CORPORATION, 10700 BREN ROAD WEST, MINNETONKA, MN, 55343, US

(21) Appl. No.: 11/627,459

(22) Filed: Jan. 26, 2007

Related U.S. Application Data
(60) Provisional application No. 60/762,697, filed on Jan. 27, 2006.

(51) Int. Cl. A61B 19/00; A61F 5/37

(52) U.S. Cl. 128/849

(57) ABSTRACT

Improved disposable surgical drapes and surgical methods for isolating the surgical field from the patient's penile and scrotal skin to prevent any contact of implantable medical device components, the surgical instruments, and the surgeon's gloved hand with the patient's skin during a device implantation or a surgical procedure are disclosed. A surgical drape comprises a sheet of drape material bounded by sheet edges adapted to lie over the patient's skin and an elongated penile sheath extending from a sheath attached end extending from the sheet to a sheath free end. The sheath may be grasped during implantation surgery to manipulate the penis without making direct contact with the penile skin. The surgical drape further comprises a scrotal skin contact portion adjacent the sheath attached end of a material capable of being penetrated in making an incision of the scrotal skin, whereby contact with the scrotal skin is avoided.

### RELATED APPLICATIONS

This application claims priority to U.S. Provisional Application Ser. No. 60/762,697 filed Jan. 27, 2006, the entire content of which is incorporated herein by reference.

### TECHNICAL FIELD

The present invention relates generally to the field of surgery, and more particularly to an improved surgical drape and surgical method for isolating the surgical field from the patient's penile and scrotal skin during performance of a surgical procedure through a scrotal skin incision, e.g., the implantation of an implantable medical device or repair of a medical condition, the surgical drape minimizing contact of the medical device, the surgical instruments, and the surgeon's gloved hand with the patient's skin during the procedure.

### BACKGROUND

Surgical drapes are commonly used to cover a patient's body during a surgical procedure involving making an incision through the patient's skin and subcutaneous tissues to access a particular site of interest. Certain procedures performed on male patients involve making an incision through scrotal skin into the interior of the scrotum. For example, surgical procedures employed in alleviating a urethral stricture or the implantation or extraction of certain implantable medical devices involve making an incision through the scrotal skin. Typical implantable medical devices implanted through scrotal skin incisions and surgically created or accessed subcutaneous cavities include urethral incontinence control devices and penile prostheses for enabling penile erection.

As described in my U.S. Pat. No. 7,066,878, various types of implantable penile prostheses are available for treatment of erectile dysfunction, and various specialized tools exist for implanting such prostheses. Inflatable penile prostheses are also disclosed in U.S. Pat. Nos. 3,954,102, 4,424,807, 4,537,183, and 5,263,981. An inflatable unitary penile prosthesis disclosed in U.S. Pat. No. 4,590,927 comprises a tubular enclosure having a distal portion that includes a pump, a medial portion including a pressurizable chamber that contains an internal tubular, substantially non-distensible portion and a concentric tubular sleeve, and a proximal portion defining a fluid reservoir therewithin.

The inflatable penile prosthesis disclosed in the '878 patent corresponds to the AMS 700™ inflatable penile prosthesis sold by AMS Research, Corp., which comprises three components that are filled with sterile saline fluid and assembled during the course of the implantation procedure. The AMS 700™ series penile prostheses include the Ultrex model, which imparts both penile girth and distal expansion, and the CX model that imparts girth expansion only. The CX model is most applicable for patients with scar tissue or those with a tendency for penile curvature upon tumescence. An InhibiZone antibiotic surface treatment (consisting of rifampin and minocycline hydrochloride) is applied to the components of the AMS 700 series penile prostheses to decrease the incidence of infection.

The typical three-piece penile prosthesis includes a pair of cylinders, a pump, a reservoir, and interconnecting tubing. The cylinders are inflatable and are each implantable in one of the corpus cavernosa to extend side-by-side within the penis. The pump is adapted to be implanted in the scrotum and is typically connected to the cylinders through tubing extending from points near one end of each cylinder. The reservoir is also connected by a length of tubing to the pump. The spherical abdominal reservoir is implanted in the pelvic region near the urinary bladder within the abdomen. The patient can manipulate the pump in order to pump fluid from the reservoir chamber into the cylinder chambers to inflate the cylinders and achieve an erection. Similarly, the patient can manipulate the pump to deflate the cylinders and refill the reservoir.

The three-component penile prostheses have proven to be most satisfactory because they produce the most natural appearing phallus in the inflated as well as the deflated states. They also provide good rigidity even for larger penises and provide good flaccidity for social dress. In the flaccid state, pressure against the corpora and tunica albuginea is also alleviated, which is helpful for patients suffering diabetes, previous implant extrusion, or previous implant infections.

The penile prostheses of these types is supplied as a kit containing the various components, including the cylinders, pump, and reservoir, with lengths of tubing attached to or attachable to each of the components. In the course of implanting the prosthesis, the surgeon introduces the kit into the sterile field and fills each of the components separately with sterile pressurizing liquid through the attached tubing, shortens the tubing to the correct length, if necessary, and then connects the components into a closed system using tube connectors as shown in the U.S. Pat. No. 4,537,183, for example. The components of the kit and the pressurizing fluid can be supplied sterile by the manufacturer of the system, but because of the surgeon's need to fill and assemble the system the sterility of the final closed system cannot be assured.

Penile prosthesis infection, occurring in about 1% to 5% of penile prosthesis implantation procedures, is a serious post-operative complication. Common pathogens or organisms that cause these infections include Staph bacteria and fungi that are implicated in about 70% of penile-prosthesis infections, can remain on the patient's skin despite rigorous and lengthy skin preparation. Some bacterial species, such as Staphylococcus epidermidis, can lie dormant for as long as 2 years before causing clinical signs of infection. Fungal infections, which constitute about 10% of these penile prosthesis infections, necessitate removal of the prosthesis and prolonged anti-fungal treatment. Risk factors for infection include prolonged hospital stay, diabetes mellitus, concomitant surgical procedures such as artificial sphincter implantation and circumcision, spinal cord injury, paraplegia, and the use of additional foreign bodies during implantation. None of these conditions or circumstances is contraindications to implantation but rather emphasize increased attention to infection prevention.

In the above-described procedure, the patient is typically draped with disposable surgical drapes that simply comprise rectangular sheets of drape material that may have an adhesive layer on the surface applied to the skin. Such drapes are not conformable to the pundendal region, particularly the penis, scrotum and perineum. The drapes are arranged to cover the patient's abdominal region leaving the scrotum and/or penis exposed when incisions and cavities are made and the prosthesis components are inserted into the cavities. The skin overlying the penis and scrotum is necessarily exposed. The surgeon's gloved hands, the instruments, and the prosthesis components come into contact with that exposed skin. The exposed skin is scrubbed for about 10 minutes with an antiseptic soap-water mixture and then painted with antiseptic to minimize the risk of infection by the above-described pathogens or organisms.

It has been proposed to provide specialized isolation of the surgical field during penile prosthesis implantation to reduce penile prosthesis infection complications or other procedures related to the genitalia in U.S. Pat. Nos. 3,862,362, 4,471,769 4,903,710, and 4,950,222. A specialized method of sterilizing and packaging a penile prosthesis is disclosed in U.S. Pat. No. 5,101,813.

Generally speaking, surgical drapes shaped to fit over the patient's abdomen and legs and having at least one fenestration or window through the sheet material to access the genitalia, are disclosed in the '362, '769, and '710 patents. The windows are typically formed as slits or circular openings through highly elastic sheet material, e.g., Kraton® polymer sheet material, so that they are expandable to enable insertion of the surgeon's gloved hands or instruments or the male genitalia therethrough. In the '222 patent, the drape is formed as part of the wall of an inflatable isolator providing a chamber for filtered air to minimize air-borne contamination of the surgical field. The patient's genitalia are drawn into the chamber through an expandable window in the drape. It is necessary to introduce the penile implant prosthesis and instruments used in the procedure into the chamber.

In these approaches, the skin overlying the penis and scrotum remains exposed, and contact with skin-borne pathogens and organisms leading to possible prosthesis infection may still occur.

During a penile prosthesis implantation procedure, the surgeon estimates an appropriate size of the penile cylinders. Correct sizing of the inflated cylinders if very difficult and crucial to optimize patient satisfaction. In order to size the cylinders to the penis, the surgeon relies on manual stretching of the flaccid penis, to obtain an initial measurement of the cylinders to be implanted. Following implantation of the cylinders, the surgeon examines the penis (shaft and glans) in the erect/inflated and flaccid/deflated state to confirm that the penile cylinders are appropriately sized. The cylinders are inflated and deflated by manipulating the pump, which at first is outside of the patient and subsequently positioned in the scrotum. After pump implantation, the cylinders also need to be inflated by manipulating the pump through the scrotal skin. If the cylinders are incorrectly sized, they must be removed and replaced with more appropriately sized cylinders. The pump and reservoir may also need to be removed and/or repositioned. The components of the penile prosthesis are likely to be contaminated if the component or the surgeon's gloves contact the patient's skin in the process of initially introducing, removing and reintroducing or repositioning the components.

Thus, a need remains for an improved surgical drape and method of more completely isolating the skin of a male patient from contact with the penile prosthesis, the surgical instruments used in the procedure, and the surgeon's gloved hands during the surgical implantation procedure.

In addition, there is an apparently unrecognized need for an improved surgical drape and method of more completely isolating the skin of a male patient from contact with components of other implantable medical devices, the surgical instruments used in the procedure, and the surgeon's gloved hands during the surgical implantation procedure.

### SUMMARY

The preferred embodiments of the present invention incorporate a number of inventive features that address the above-described problems that may be combined as illustrated by the preferred embodiments or advantageously separately employed.

In accordance with one aspect of the invention, an improved surgical drape covers one or both of the penis and scrotum during the course of the implantation of an implantable medical device or performance of a surgical procedure through a scrotal skin incision.

In preferred embodiments, the surgical drape for covering a patient during a surgical procedure involving making a scrotal skin incision comprises a sheet of drape material bounded by sheet edges adapted to lie over the patient's skin to extend superior, inferior and lateral to the patient's penis and scrotum, and an elongated penile sheath extending from a sheath attached end extending from the sheet to a sheath free end. The penile sheath is shaped and sized to receive the patient's penis within the lumen of the penile sheath, whereby the sheath may be grasped during surgery to manipulate the penis without making direct contact with the penile skin.

In further preferred embodiments, the surgical drape further comprises a scrotal skin contact portion adjacent the sheath attached end of a material capable of being penetrated in making an incision of the scrotal skin. The incision through the scrotal skin contact portion and the underlying scrotal skin and subcutaneous tissue may be made to enable accessing a site of a surgical procedure or implantation or explantation of components of an implantable medical device through the scrotal skin incision without contacting the scrotal skin.

In certain embodiments, the surgical drape may further comprise a skin adhesive layer on a side of the scrotal skin contact portion adapted to be placed over the scrotum to adhere the scrotal skin contact portion to the scrotal skin.

Preferably the sheath is closed at the sheath free end, but can be cut open or penetrated to access the penis when necessary in the course of any surgical procedure.

The sheet of drape material may be transparent at least in part and compliant particularly in the penile sheath and/or the scrotal skin contact portion whereby the penis and/or scrotum may be visualized and manipulated to a variety of positions and states in the course of making tissue incisions and tunneling through tissue to create spaces for the components of an implantable medical device.

In use of a preferred embodiment, the surgical drape is preferably applied over the patient's exposed skin and any conventional drapes positioned on the patient's body, the penis inserted into the penile sheath lumen through a sheath attached end opening, and the scrotal skin contact portion overlying the scrotum. The sterile gloves employed in positioning the surgical drape may then be replaced to continue with the implantation procedure.

In certain procedures, a urethral catheter, e.g., a Foley catheter, may be placed before the penis is inserted into the sheath to block the bladder. In one approach, the Foley catheter end extending from the urethral orifice may be capped with a plug, and both the penis and the plugged Foley catheter enclosed within the sheath lumen. In another approach, the catheter may be placed before or after the penis is inserted into the sheath lumen with the catheter extending through an opening made in the sheath free end.

The surgical drape may advantageously be employed in conjunction with other instruments for stabilizing and positioning the penis and scrotum, e.g., a retractor. The retractor may be placed over or under the surgical drape.

For example, a conventional implantation kit may be utilized to fix the penis extending in a cephalid or superior direction (12 o'clock) so that a transverse or longitudinal incision may be made through the scrotal skin contact portion and the underlying scrotal skin and subcutaneous tissues. Tension may be applied to draw the skin and overlying scrotal skin contact portion material laterally to maintain and enlarge the incision and to tunnel subcutaneously.

In the course of implanting a penile prosthesis, the penile sheath may be grasped to manipulate the penis as cavities are formed and enlarged extending side-by-side through the corpus cavernosa to receive the elongated cylinders. Similarly, the scrotum may be manipulated through the scrotal skin contact portion during the steps of implanting the pump in the scrotum and the reservoir behind the pubic bone and the interconnecting the components.

Thus, contact with the scrotal skin and/or penile skin is advantageously eliminated during the implantation of the components of the penile prosthesis. While implantation of a three-component penile prosthesis is described, it will be understood that the surgical drapes and methods of the invention may advantageously be employed in implantation of any type of inflatable or non-inflatable penile prosthesis.

Moreover, it will be understood that the surgical drapes and methods of the invention may advantageously be employed in performing other procedures through a scrotal incision, e.g., accessing the male bulbar urethra to implant an inflatable cuff about the urethra and other components of a urinary incontinence prosthesis or to implant a sling extending under the urethra and through the abdomen to passively alleviate male incontinence or to perform other procedures, e.g., alleviating a urethral stricture or explanting a previously implanted medical device.

In a related embodiment, the surgical drape is modified to be used in procedures involving vaginal, rectal or perineal incisions and implantation of devices to treat urinary and fecal incontinence. In yet another embodiment, the drape is modified for gastric or abdominal incisions for related treatments. See U.S. patent Publication No. U.S. 2002-0078964 A1, published Jun. 27, 2002 (Kovac), which is herein incorporated by reference.

This summary of the invention has been presented here simply to point out some of the ways that the invention overcomes difficulties presented in the prior art and to distinguish the invention from the prior art and is not intended to operate in any manner as a limitation on the interpretation of claims that are presented initially in the patent application and that are ultimately granted.

### BRIEF DESCRIPTION OF THE DRAWINGS

These and other advantages and features of the present invention will be more readily understood from the following detailed description of the preferred embodiments thereof, when considered in conjunction with the drawings, in which like reference numerals indicate identical structures throughout the several views, and wherein:

FIG. 1 is a perspective view of the components of an exemplary penile prosthesis that may be implanted in a patient employing the surgical drapes and methods of the present invention;

FIG. 2 is a perspective view of the assembled components of the penile prosthesis of FIG. 1 filled with fluid and implanted in a patient's body;

FIG. 3 is a schematic top view of one step in the conventional procedure of implanting a penile prosthesis of the type depicted in FIGS. 1 and 2 in a patient's body utilizing a conventional surgical drape and an implantation kit;

FIG. 4 is a schematic top view of a surgical drape in accordance with one embodiment of the present invention depicting a penile sheath extending along one side of the sheet of drape material;

FIG. 5 is a side cross-section view of the surgical drape taken along lines 5-5 of FIG. 4 depicting the penile sheath and optionally the scrotal skin contact portion extending laterally from the sheet of drape material;

FIG. 6 is a schematic top view of the surgical drape of FIGS. 4 and 5 encasing the penis and overlying the scrotum disposed within retractor windows prior to making an incision; and

FIG. 7 is a schematic top view of the step illustrated in FIG. 3 with the surgical drape of FIGS. 4 and 5 encasing the penis and overlying the scrotum after making an incision through the scrotal portion of the surgical drape and the underlying scrotal skin and tissues.

It will be understood that the drawing figures are not necessarily to scale.

### DETAILED DESCRIPTION OF THE PREFERRED EMBODIMENTS

In the following detailed description, references are made to illustrative embodiments of methods and apparatus for carrying out the invention. It is understood that other embodiments can be utilized without departing from the scope of the invention.

The surgical drapes and methods of the invention may advantageously be employed in performing a variety of procedures through a scrotal incision, e.g., accessing the male bulbar urethra to implant an inflatable cuff about the urethra and other components of a urinary incontinence prosthesis or to implant a sling extending under the urethra and through the abdomen to passively alleviate male incontinence or to perform other procedures, e.g., alleviating a urethral stricture. Therefore, it will be understood that the following description of a preferred embodiment involving use of the surgical drapes and methods of the invention during the course of implantation of a penile prosthesis is not limiting.

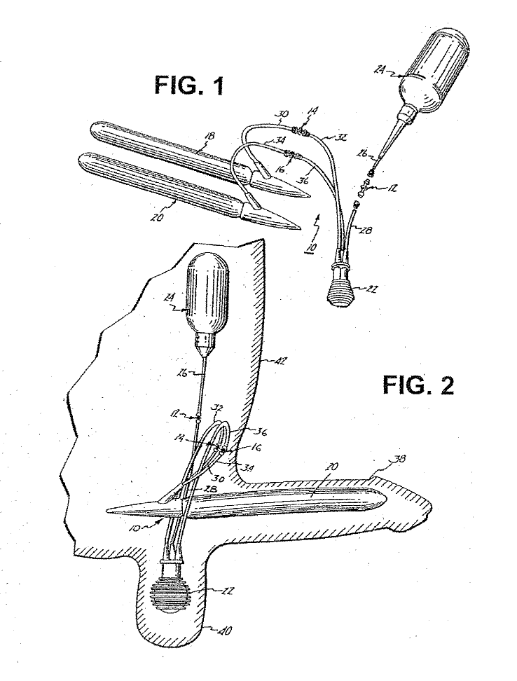

Referring to FIG. 1, an exemplary embodiment of a three-component penile prosthesis 10 disclosed in the above-referenced '183 patent is depicted (as partially assembled from the components) that may be implanted employing a surgical drape in accordance with the teachings of the present invention. The components comprise the cylinders 18 and 20, the fluid reservoir 24, the valve and pump assembly (or simply, pump) 22, and intercommunicating flexible tubes or conduits 26, 28, 30, 32, 34, and 36 and connectors 12, 14, and 16. It will be understood that in practice the configurations of the components may differ from the depicted exemplary penile prosthesis 10.

The cylinders 18 and 20 are elongated, and each includes a proximal (toward the patient's pelvis) portion, a distal (toward the glans of the penis) portion, and a medial portion. The medial portions include the cylinder fluid chambers surrounded by flexible elastic walls that can be expanded and pressurized by fluid pumped from the reservoir chamber by manual manipulation of the pump 22. The proximal portions are more rigid than the medial portions and include the junctions between the cylinders 18 and 20 and the respective and tubes 30 and 34. Each of the cylinders 18 and 20 is an enclosed hollow member formed of a very thin, e.g., 25 mils, flexible membrane. Any biocompatible synthetic material tolerated by the tissues, e.g., silicone rubber, may be used. As can be seen clearly in FIG. 1, each cylinder is of generally cylindrical shape whose opposed ends are rounded or domed. Each cylinder has sufficient volumetric capacity so that when it is inflated to the condition where the fibrous envelope surrounding the corpora cavernosa has reached the limit of its expansion, the material forming the membrane wall of the cylinder has not reached the point at which it undergoes tension, e.g., it does not begin to stretch.

As shown in FIG. 2, the penile prosthesis 10 is adapted to be surgically implanted in the body with the two cylinders 18 and 20 being disposed side-by-side within the corpus cavernosa of penis 38, the pump 22 disposed within the scrotum 40, and the reservoir 24 disposed within the abdominal cavity subcutaneous to the abdominal skin 42. The cylinders 18 and 20 are constructed to mechanically expand the corpora cavernosa until the fibrous tissue envelope becomes tense, thereby producing a functional erection without the necessity for the corpora cavernosa to be engorged with blood. The cylinders are readily deflatable to enable the penis 38 to become flaccid when an erection is no longer sought.

The cylinders 18 and 20 are placed within the respective corpora cavernosum by surgically preparing a passageways therein extending from proximal scrotal incision distally toward the glans of the penis 38. The passageways are formed by any conventional surgical techniques used for penile prosthesis implantations. The pump 22 is implanted within a cavity within the scrotum that is created by blunt dissection from the same lateral incision made in the scrotum. The reservoir 24 is implanted into a space in the abdominal cavity behind or near the patient's pubic bone that is also accessed through the same lateral scrotal incision.

In the depicted example, the connectors 12, 14, and 16 are used to attach the respective tube pairs 26-28, 30-32, and 34-36 in the course of implantation. Fewer and different forms of connectors may be employed depending upon the penile prosthesis model. In any case, the fluid chambers of the cylinders 18, 20, the pump 22, and the reservoir 24 are filled with sterile saline and the tubes clamped closed prior to implantation of each component.

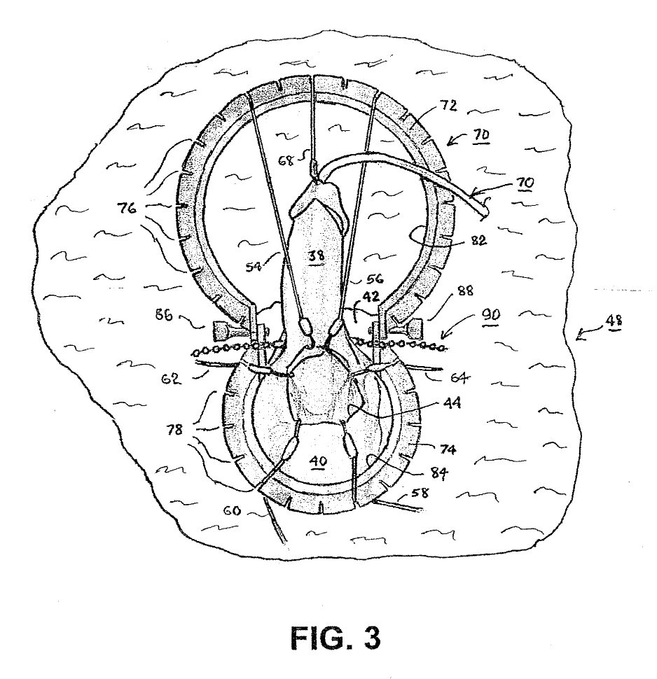

Turning to FIG. 3, it depicts one step in the conventional procedure of implanting a penile prosthesis of the type depicted in FIGS. 1 and 2 in a patient's body employing a kit of instruments, e.g., the AMS SKW™ Retractor System sold by AMS Research, Corp., that is used to stabilize the penis and the scrotal incision during steps of the procedure. The kit comprises seven blunt hooks 54, 56, 58, 60, 62, 64 and 66, one sharp hook 68, a Scott retractor 70, a beaded penile strap 90, and two baby Deavers (not shown). Further instruments for effecting implantation of the components, including those instruments disclosed in the above-referenced '878 patent may be employed in the implantation procedure.

Each of the hooks 54-68 comprises a length of line extending from it. The hourglass-shaped Scott retractor 70 comprises a pair of C-shaped frames 72 and 74 having a plurality of slots or notches 76 and 78, respectively, in the frame outer edges, the major and minor (in diameter) frames 72 and 74 defining respective major and minor (in area) retractor windows 82 and 84. The frames 72 and 74 are joined together by clamps 86 and 88. The penile strap 90 is beaded along its length and may comprise a length of elastic tubing over a central portion of its length. In use, the penile strap is extended between the joined ends of the pair of C-shaped frames 72 and 74 and fitted under tension into an opposed pair of notches 78 of the minor frame 74.

At this illustrated stage in the procedure, the patient has been prepared lying on his back, sedated, and draped with a conventional surgical drape 48 leaving the penis 38, a small area of the abdominal skin 42 near the penis 38, and the scrotum 40 exposed. The beaded penile strap 90 is attached to the retractor 70 and the retractor-strap assembly is placed onto the surgical drape 48. The penis 38 is folded over the beaded penile strap 90 to extend into the major window 82, and the scrotum 40 is disposed centered in the minor retractor window 84. The sharp hook 68 is hooked into the penis glans, and tension is applied to the hook line fitted into one of the notches 76 to stretch the penis 38 extending toward 12 o'clock in the major retractor window 82. As depicted, a Foley catheter 70 has been advanced through the urethral orifice, through the patient's urethra, and into the bladder. In a conventional approach, the Foley catheter balloon is inflated in the bladder, and the catheter free end may be attached to a drainage bag.

In addition, the surgeon has made a transverse incision 44 in the high scrotum through the subcutaneous tissues, and the scrotal skin has been retracted in the superior, inferior and lateral directions. The incision 44 is moved onto the penis 38, and the widened incision 44 is secured by blunt hooks 54 and 56 fixed to the incision edge and drawn in the 11 o'clock and 1 o'clock directions by hook lines secured to a selected pair of notches 76. Similarly, the incision 44 is drawn in the inferior direction, and the widened incision 44 is secured by blunt hooks 58 and 60 fixed to the incision edge and drawn in the 5 o'clock and 7 o'clock directions by hook lines secured to a selected pair of notches 78. The incision 44 is also drawn laterally and secured by blunt hooks 62 and 64 fixed to the incision edge and drawn in the 9 o'clock and 3 o'clock directions by hook lines secured to a selected pair of notches 76.

The remaining steps of implantation include performing a corporotomy into the corpora, tunneling into and dilating the corpus cavernosa, fitting the tubular cylinders 18 and 20 into the corpus cavernosa, and bluntly dissecting tissues to implant the reservoir 24 in the abdomen and the pump 22 in the scrotum 40 take place while the penile and scrotal skin are exposed. The penis 38 is manually grasped and manipulated by the surgeon during dilation of the corpus cavernosa and insertion of the tubular cylinders 18 and 20 into the corpus cavernosa. At times, the reservoir 24 and pump 22 may be left lying on the exposed scrotal skin within the window 84. As noted above, there is a danger of introducing a pathogen or organism into the incision 44 leading to infection of the implantation sites.

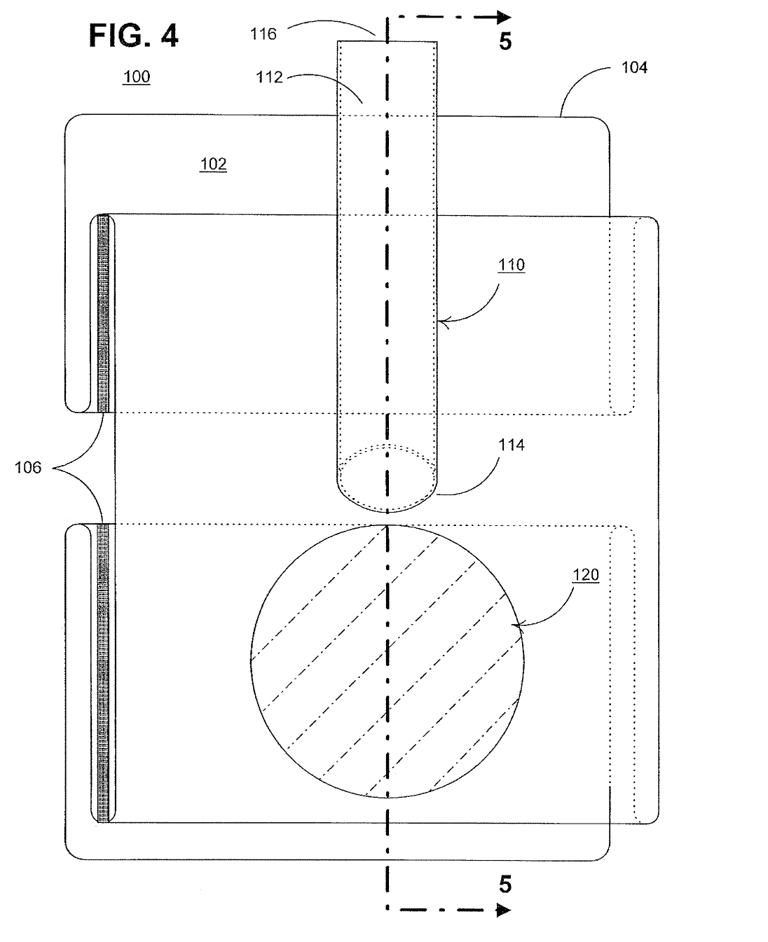

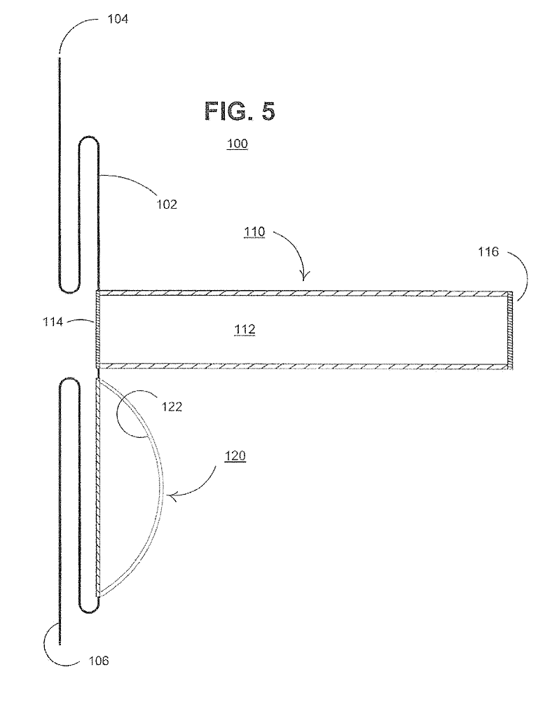

In accordance with the present invention, a surgical drape 100 depicted in FIGS. 4 and 5 is provided that substantially covers the penile and scrotal skin and any exposed abdominal skin during such a penile prosthesis implantation procedure and in other procedures involving scrotal incisions. The scrotal incisions may be anywhere in the scrotal skin and extend in any direction.

In preferred embodiments, the surgical drape 100 for covering the patient during the surgical procedure comprises a sheet 102 of drape material bounded by sheet edges 104 adapted to lie over the patient's skin (and any conventional drapes applied to the abdominal skin and legs) to extend superior, inferior and lateral to the patient's penis 38 and scrotum 40. One or more strip 106 of skin adhesive may be provided extending along the periphery of the skin or other drape contacting side of the sheet 102 to stabilize the surgical drape edges 104 during a surgical procedure. For convenience of illustration, the sheet 102 is depicted as relatively planar. It will be understood that the sheet 102 may be three-dimensional in portions to conform to the body anatomy in the abdominal, pelvic, inguinal and femoral regions, including the perineum and surrounding areas. The surgical drape 100 comprises specific structures described below that conform to the penis and scrotum in the pudendal region.

An elongated penile sheath 110 having a sheath lumen 112 extends from a sheath attached end 114 to a sheath free end 116. At least the sheet attached end is open to the sheath lumen 112. The penile sheath 110 is shaped and sized to receive the patient's penis 38 within the lumen 112 of the penile sheath 110, whereby the sheath 110 may be grasped during implantation surgery to manipulate the penis 38 without making direct contact with the penile skin. The sheath length and lumen diameter is selected to facilitate performance of the procedure, e.g., to accommodate a penis 38 that is enlarged during the steps of forming the cylinder receiving cavities in the corpus cavernosa and inserting the cylinders 18 and 20 into the cavities. The sheath length may be any suitable length that will accommodate the penis and instruments, e.g., a length of a Foley catheter inserted into the urethra.

The surgical drape material is preferably transparent at least in the penile sheath 110 and a portion of sheet 102 overlying the scrotum 40, so that the penis 38 and scrotum 40 may remain under visual observation during the procedure. The drape material is also preferably highly compliant and tear resistant, at least in the penile sheath 110 and a portion of sheet 102 overlying the scrotum 40, so that the material remains fitted about or against the penis 38 and scrotum 40 while they are manipulated. Suitable materials for sheet 102 and penile sheath 110 comprise a latex free, polymeric, substantially transparent film that may include an anti-microbial agent, of the types utilized in the Steri-Drape™ surgical drapes available from Minnesota Mining and Manufacturing Company.

In further preferred embodiments, the surgical drape 100 further comprises a scrotal skin contact portion 120 adjacent the sheath attached end 114 of a material capable of being penetrated in making an incision of the scrotal skin. The incision through the scrotal skin contact portion 120 and the underlying scrotal skin and subcutaneous tissue may be made to enable implantation of components of the penile prosthesis 10 through the scrotal skin incision without contacting the scrotal skin. The scrotal skin contact portion 120 may have a hemispherical shape that extends away from the sheet 102 in the same direction as the penile sheath 110 in order to accommodate the scrotum when the sheet 102 is applied against the patient's skin. The boundary the scrotal skin contact portion 120 may be circular as depicted or may have any other suitable shape. The size and area of the scrotal skin contact portion 120 may be any suitable size preferably exceeds the typical size of the adult male scrotum. As described below, the scrotal skin contact portion may extend away from the scrotum and over certain medical instruments.

The material selected for scrotal skin contact portion 120 is also preferably transparent, highly compliant and tear resistant so that the material remains fitted about or against the scrotum 40 while it is manipulated and the incision is made. The tear resistance is selected to avoid accidental enlargement of the intentionally made incision through the scrotal skin contact portion 120 and underlying scrotal skin and subcutaneous tissues. In certain embodiments, the surgical drape 100 may further comprise a skin adhesive layer 122 on a side of the scrotal skin contact portion 120 adapted to be placed over the scrotum 40 to adhere the scrotal skin contact portion 120 to the scrotal skin.

Suitable materials for portion 120 comprise an incise drape material utilized in the Steri-Drape™ Incise film surgical drapes available from Minnesota Mining and Manufacturing Company and similar drapes available from T. J. Smith and Nephew Ltd. An incise material is usually an elastic, substantially transparent, polymeric, latex free, film with an adhesive on one side which is in turn covered with a release liner. The release liner is removed at the time that the adhesive layer is applied to the patient's skin, the scrotal skin in this instance.

The penile sheath free end 116 may be open or closed depending on the requirements of the procedure. In this embodiment, the penile sheath free end 116 is closed to provide maximal isolation of the penile skin during implantation of implantable medical device components through the scrotal incision. Direct access to the penis glans and the external urethral orifice may be required during the surgical procedure. In certain instances, the closed sheath free end 116 may simply be cut open or penetrated at the appropriate point in the procedure.

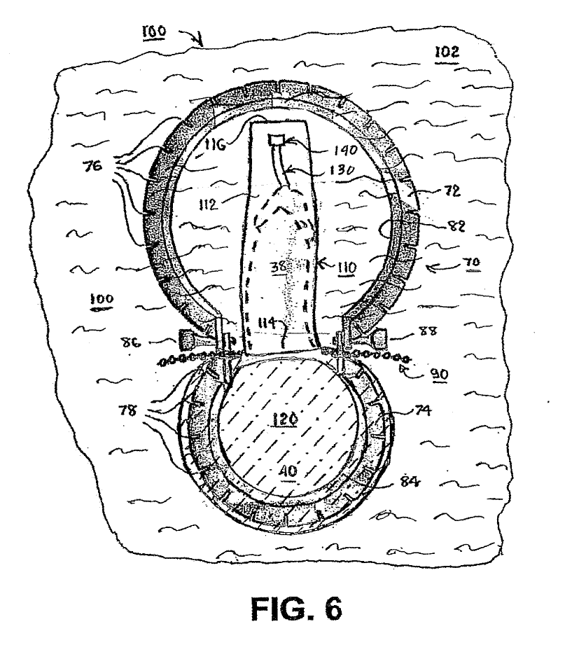

In use of the preferred embodiment depicted in FIG. 6, the surgical drape 100 is preferably applied over the patient's skin or another conventional drape applied against the patient's skin. Methods of preparing and draping a patient with surgical drapes culminating with an incise drape material over the site to be incised is disclosed in U.S. Pat. No. 5,803,086, for example. In the present invention, the adhesive layer 122 of the scrotal skin contact portion 120 is applied against the scrotal skin. The penis 38 is inserted through the opening of the sheath attached 114 into the penile sheath lumen 112. The scrotal skin contact portion 120 is applied overlying the scrotum 38 and may extend peripherally under or over the minor frame 74.

One catheterization approach is depicted in FIG. 6 that has been completed prior to insertion of penis 38 in sheath lumen 112. In this approach, a urethral catheter 130, e.g., a Foley catheter, is placed before the penis 38 is inserted into the sheath lumen 112 to catheterize the bladder. The open lumen at the Foley catheter end extending from the urethral orifice is obstructed by a stopper 140, e.g., a Foley catheter plug, and both the penis 38 and the plugged Foley catheter 130 are enclosed within the sheath lumen 112. The Foley catheter body may have to be shortened to fit into the sheath lumen 112. In the conventional approach, the catheter 130 may be placed before or after the penis 38 is inserted into the sheath lumen 112 with the catheter 130 is extended through an opening made in the sheath sidewall or free end 116 to a bag attached to the catheter free end. The depicted approach allows the penis 38 and sheath 110 to be more freely manipulated, and the bag does not act to inadvertently pull or drag on the sheath 110 and penis 38.

The sterile gloves employed in positioning the surgical drape 100 as depicted in FIG. 6 may be replaced to continue with the surgical or implantation procedure.

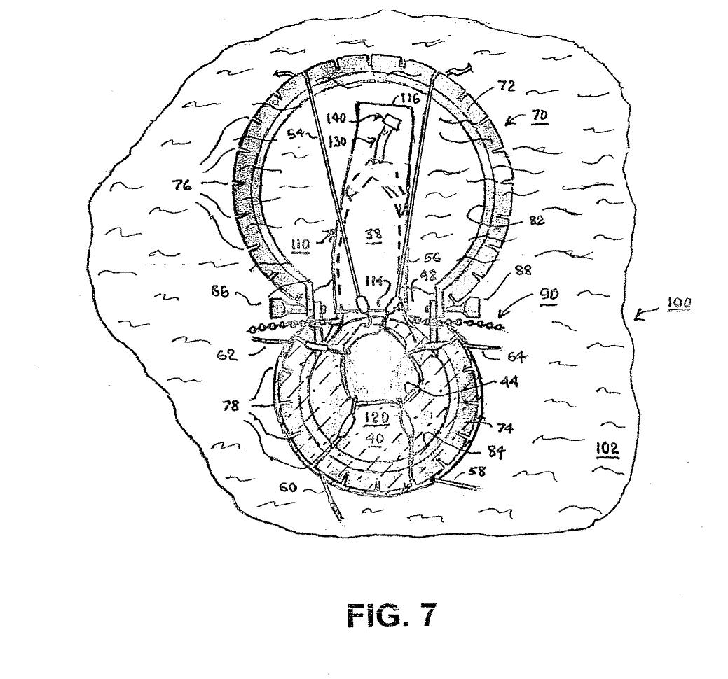

As shown in FIGS. 6 and 7, the conventional implantation kit described above with respect to FIG. 3 may be utilized with the surgical drape 100. The retractor 70 may be positioned over or under the surgical drape 100. In one approach, the retractor 70 is positioned over the skin or an underlying conventional surgical drape, and the surgical drape 100 is placed over the retractor 70 as indicated by wavy lines. The catheterized (or un-catheterized) penis 38 is extended into the sheath lumen 112 as the drape is applied, and the drape material extends over the major 72. The scrotal skin contact portion 120 is applied against the scrotal skin and over the minor frame 74 as indicated by the broken lines. The material of the scrotal skin contact portion 120 may be tucked around the minor frame 74. The material of the drape 100 is flexible enough to allow the lines of the hooks 54-68 to be inserted over the drape material and into the grooves or slits in the major and minor frames 72 and 74.

In FIG. 7, the penis 38 is extended in a cephaled or superior direction (12 o'clock) and the hook 68 may or may not be affixed to the penis 38 through the sheath 110 in the manner depicted in FIG. 3. The transverse (or longitudinal) incision 44 may be made through the scrotal skin contact portion 120 and the underlying scrotal skin and subcutaneous tissues. Tension may be applied using the blunt hooks 56-64 to draw the skin and overlying scrotal skin contact portion 120 laterally to maintain and enlarge the incision 44. The penile sheath 110 may be grasped to manipulate the penis 38 as cavities are formed and enlarged extending side-by-side through the corpus cavernosa to receive the elongated cylinders 18 and 20. Similarly, the scrotum 40 may be manipulated through the scrotal skin contact portion 120 during the steps of implanting the pump 22 in the scrotum 38 and the reservoir 24 behind the pubic bone and the interconnecting the components.

Manipulation of the penis is necessary during the course of the penile implantation procedure to estimate the appropriate size of the cylinders 18 and 20 to be implanted. The selected cylinders 18 and 20 may prove to be too long or too short when they are implanted in the corpus cavernosa and inflated. The cylinders 18 and 20 are then removed and a more appropriately sized set of cylinders 18 and 20 are implanted in the corpus cavernosa and inflated. The isolation of the penile and scrotal skin afforded by the surgical drape of the present invention lessens the probability of infection that currently exists when the penis and scrotum come into contact with the components of the penile prosthesis during the procedure.

Thus, contact with the scrotal skin and/or penile skin is advantageously eliminated during the implantation of the components of the penile prosthesis 10. While implantation of a three-component penile prosthesis 10 is described, it will be understood that the surgical drapes and methods of the invention may advantageously be employed in implantation of any type of inflatable or non-inflatable penile prosthesis.

As noted above, it will be understood that the surgical drape 100 and methods of the invention may advantageously be employed in performing other procedures through the scrotal incision 44. Prosthetic urethral sphincters have been proposed and made clinically available that are adapted to be implanted around the urethra and inflated to close the urethra to prevent incontinence and deflated to release the urethra and allow urination as disclosed in U.S. Pat. Nos. 4,412,530, 4,222,377, 4,878,889, 5,562,598, 6,749,566, and 7,011,622 for example. Such urethral sphincters comprise an inflatable urethral cuff, a balloon reservoir/pressure source, and a pump. In males, the cuff is typically implanted around the bulbous urethra, and the pump is typically implanted within the scrotum. The urethral cuff functions similarly to a blood pressure cuff to constrict the urethra sufficiently to avoid incontinence. The above-described procedure for using the surgical drape 100 to isolate the penile and scrotal skin and making the scrotal incision can be followed to gain access to the male bulbar urethra to implant an inflatable cuff about it. Similarly, the pump and balloon reservoir/pressure source may be implanted in the scrotum and behind the abdominal wall, respectively, while the scrotal and penile skin are isolated.

Moreover, it will be understood that the surgical drape 100 and methods of the invention may advantageously be employed in performing other procedures through the scrotal incision 44 adapted as necessary to access subcutaneous tissues and organs to perform surgical repairs or treatments of the same, e.g., alleviating a urethral stricture.

All patents and publications referenced herein are hereby incorporated by reference in their entireties.

It will be understood that certain of the above-described structures, functions and operations of the above-described preferred embodiments are not necessary to practice the present invention and are included in the description simply for completeness of an exemplary embodiment or embodiments. It will also be understood that there may be other structures, functions and operations ancillary to the typical surgical procedures that are not disclosed and are not necessary to the practice of the present invention.

In addition, it will be understood that specifically described structures, functions and operations set forth in the above-referenced patents can be practiced in conjunction with the present invention, but they are not essential to its practice.

It is therefore to be understood, that within the scope of the appended claims, the invention may be practiced otherwise than as specifically described without actually departing from the spirit and scope of the present invention.

1. A surgical drape for covering the genitalia of a patient during a surgical procedure comprising:
a sheet of drape material bounded by sheet edges adapted to lie over the patient's skin to extend superior, inferior and lateral to the patient's penis and scrotum; and
an elongated penile sheath extending from a sheath attached end extending from the sheet to a sheath free end, the penile sheath shaped and sized to receive the patient's penis within the lumen of the penile sheath, whereby the sheath may be grasped during surgery to manipulate the penis without making direct contact with the penile skin.

2. The surgical drape of claim 1, further comprising a scrotal skin contact portion adjacent the sheath attached end of a material capable of being penetrated in making an incision of the scrotal skin, whereby an incision through the scrotal skin contact portion and the underlying scrotal skin and subcutaneous tissue may be made to enable implantation of components of an implantable medical device or treatment of the patient through the scrotal skin incision without contacting the scrotal skin.

3. The surgical drape of claim 2, further comprising a skin adhesive layer on a side of the scrotal skin contact portion adapted to be placed over the scrotum to adhere the scrotal skin contact portion to the scrotal skin.

4. The surgical drape of claim 2, wherein the penile sheath free end is closed.

5. The surgical drape of claim 2, wherein the surgical drape is substantially transparent at least in the region of the penile sheath and the scrotal skin contact portion.

6. The surgical drape of claim 1, wherein the penile sheath free end is closed.

7. The surgical drape of claim 6, wherein the penile sheath is formed of a substantially transparent material.

8. The surgical drape of claim 1, wherein the penile sheath is formed of a substantially transparent material.

9. A surgical drape for covering a patient during a surgical procedure of implanting components of an implantable medical device in the patient comprising:
a sheet of drape material bounded by sheet edges adapted to lie over the patient's skin to extend superior, inferior and lateral to the patient's penis and scrotum; and
an elongated penile sheath extending from a sheath attached end extending from the sheet to a sheath free end, shaped and sized to receive the patient's penis within the lumen of the penile sheath, whereby the sheath may be grasped during implantation surgery to manipulate the penis without making direct contact with the penile skin.

10. The surgical drape of claim 1, further comprising a scrotal skin contact portion adjacent the sheath attached end of a material capable of being penetrated in making an incision of the scrotal skin, whereby an incision through the scrotal skin contact portion and the underlying scrotal skin and subcutaneous tissue may be made to enable implantation of components of the implantable medical device through the scrotal skin incision without contacting the scrotal skin.

11. A method of preparing a male patient for a surgical procedure comprising:
providing a surgical drape comprising a sheet of drape material bounded by sheet edges and having an elongated penile sheath extending from a sheath attached end extending from the sheet to a sheath free end;
preparing the patient's skin;
draping the surgical drape over the patient's skin to extend superior, inferior and lateral to the patient's penis and scrotum; and
inserting the patient's penis into the penile sheath enabling the sheath to be grasped during surgery to manipulate the penis without making direct contact with the penile skin.

12. The method of claim 11, wherein the providing step comprises providing the surgical drape having a scrotal skin contact portion adjacent the sheath attached end of a material capable of being penetrated in making an incision of the scrotal skin and further comprising:
making an incision through the scrotal skin contact portion and the underlying scrotal skin and subcutaneous tissue to enable passage through the scrotal skin incision without contacting the scrotal skin.

13. The method of claim 12, wherein the providing step comprises providing a skin adhesive on the side of the scrotal skin contact portion adapted to be placed over the scrotum to adhere the scrotal skin contact portion to the scrotal skin.

14. The method of claim 12, wherein:
the providing step comprises providing the penile sheath with a sheath lumen and a closed sheath free end; and
the inserting step comprises:
placing a catheter having a catheter lumen through the urethra orifice, the urethra and into the bladder;
plugging the catheter lumen of the catheter end extending from the urethra orifice; and
inserting the catheter and penis into the sheath lumen.

15. The method of claim 12, wherein the surgical drape is substantially transparent at least in the region of the penile sheath and the scrotal skin contact portion.

16. The method of claim 11, wherein the providing step comprises providing the penile sheath with an open sheath attached end and a closed sheath free end and further comprising:
opening the sheath free end at least during certain steps of the surgical procedure.

17. The method of claim 16, wherein the penile sheath is formed of a substantially transparent material.

18. The method of claim 11, wherein the penile sheath is formed of a substantially transparent material.

19. The surgical drape of claim 1, wherein the drape material and elongated sheath are adapted to lie over an area selected from the group consisting of the pelvic area, perineum, the rectum/anus, abdomen and the stomach.
